(19) United States
(12) Patent Application Publication
(10) Pub. No.: US 2021/0154047 A1
(43) Pub. Date: May 27, 2021

(54) ULTRA-THIN DRUG PUMP

(71) Applicants: SOGANG UNIVERSITY RESEARCH FOUNDATION, Seoul (KR); NEO VISION CO., LTD., Yongin-si (KR)

(72) Inventors: Jung Yul Park, Seoul (KR); Cong Wang, Seoul (KR); Jung Wook Kim, Seoul (KR)

(73) Assignees: SOGANG UNIVERSITY RESEARCH FOUNDATION, Seoul (KR); NEO VISION CO., LTD., Yongin-si (KR)

(21) Appl. No.: 16/698,856

(22) Filed: Nov. 27, 2019

(51) Int. Cl. A61F 9/00; A61M 5/142

(52) U.S. Cl. CPC A61F 9/0017; A61M 2205/8287; A61M 5/142

(57) ABSTRACT

An ultra-thin drug pump may include a micro-body including a drug chamber, an intermediate outlet formed on one upper side, and an aperture formed on the other upper side; a first membrane covering the aperture; a second membrane covering the intermediate outlet and including a drug outlet formed at a position spaced apart from the intermediate outlet; and a magnetic driving part formed on the first membrane to face the drug chamber, and can release a predetermined amount of the drug by an external magnetic field.

### BACKGROUND

### 1. Technical Field

The present disclosure relates to a drug pump that can be supplied in vivo periodically or by an external command, and more particularly, to a drug pump that can be manufactured in an ultra-thin thickness to be installed into a contact lens.

### 2. Description of the Related Art

Korean Patent No. 10-1839846 discloses an in vivo implantable drug pump driven by an external magnetic field. The in vivo implantable drug pump is for treating retinopathy such as age-related macular degeneration (AMD), retinal vein occlusion (RVO), diabetic macular edema (DME), posterior uveitis, and the like, and is inserted directly into the vitreous cavity to supply a drug periodically or by an external manipulation.

In the above-issued patent, a membrane, a micro-body, a resistant membrane, and a magnetic driving part are stacked up and down, which makes it difficult to apply to an equipment having a thin thickness such as a lens.

### SUMMARY

The present disclosure provides an ultra-thin drug pump that can be installed or inserted into a lens or the like.

The present disclosure provides an ultra-thin drug pump that can be implanted or stayed at a specific position in the body and can control nanocomposite-drug release as needed.

The present disclosure provides an ultra-thin drug pump that can continuously release a drug at least twice, if necessary, while staying at a specific position in the body without being limited to only one-time release of a drug at the specific position.

In order to achieve the above objects of the present disclosure, according to an exemplary embodiment of the present disclosure, the ultra-thin drug pump may include a micro-body including a drug chamber, an intermediate outlet formed on one upper side, and an aperture formed on the other upper side; a first membrane covering the aperture; a second membrane covering the intermediate outlet and including a drug outlet formed at a position spaced apart from the intermediate outlet; and a magnetic driving part formed on the first membrane to face the drug chamber.

The magnetic driving part may move in one direction by an external magnetic field, and the first membrane pressurizes a drug in the drug chamber, and then some of the drug may be moved between the second membrane and an upper surface of the micro-body through the intermediate outlet, and the second membrane may be partially deformed to release the drug through the drug outlet.

The aperture and the intermediate outlet may be formed on an upper surface of the micro-body, and a drug in the drug chamber may move horizontally and be released through the intermediate outlet and the drug outlet.

In particular, according to one embodiment of the present disclosure, since the aperture and the intermediate outlet are formed on the same surface, the first membrane and the second membrane may be formed with the same membrane. Although the first membrane and the second membrane may be formed independently, such simultaneous formation with one membrane may simplify the structure and facilitate the manufacturing process.

However, since the first membrane is required to move toward the drug chamber and at the same time the second membrane is required to move oppositely away from the drug chamber, when they are formed with the same membrane, the pumping action may not be performed smoothly by offsetting each other's movement. In order to overcome this problem, according to one embodiment of the present disclosure, one membrane may be used to form a fixed area in which a portion of the membrane between the aperture and the intermediate outlet is secured to an upper surface of the micro-body to block a membrane deformation at the aperture and a membrane deformation at the intermediate outlet.

The micro-body may be formed using Ostemers™ 322 crystal clear, polydimethylsiloxane (PDMS), or the like. To this end, the micro-body may be formed using a multiple layer. Specifically, the micro-body may be provided by stacking a first layer forming a bottom; a second layer forming an outermost sidewall (including an intermediate support) for the drug chamber on the first layer; a third layer providing an aperture and an intermediate outlet on the second layer; one integrated membrane provided on the third layer; and a fourth layer covering a fixed area, a periphery of the aperture, and a periphery of the intermediate outlet on the integrated member.

The membrane may be secured to the micro-body by the third and fourth layers. In the fixed area, the membrane may be secured by the third and fourth layers such that deformation of a portion of the membrane around the aperture is not transferred to the membrane around the intermediate outlet, and vice versa.

In the third layer, a non-bonded region may be formed around the intermediate outlet. The non-bonded region may prevent the membrane from intermingling with the periphery of the intermediate outlet. To this end, a plurality of grooves may be formed in an upper surface of the third layer in contact with the membrane corresponding to the non-bonded region. When the membrane and the third layer are formed of Ostemers or PDMS, a bottom surface of the membrane and the upper surface of the third layer corresponding to the non-bonded region may be masked and exposed to oxygen plasma.

It is very important to find a valid dimension overall as it may be required to form a thickness to about 500 μm or less as a whole. A releasing amount of the drug may be determined by a volume pressurized while the membrane moves under exposure to a magnetic field. For this purpose, a magnetic driving part may be preferably located at a center of the diameter of the aperture. Assuming that the aperture and the magnetic driving part are circular, the diameter of the magnetic driving part relative to the diameter of the aperture may be in a range of about 0.42 to 0.60 to achieve the best releasing amount.

The magnetic driving part may be provided by stirring PDMS and magnetic nanoparticles, and the magnetic driving part may be formed to face the drug chamber at a bottom of the first membrane to keep the area exposed to the outside to a minimum.

The ultra-thin drug pump of the present disclosure can be implanted at a specific position in a body to control the release of a drug when necessary, wherein the drug pump cannot be limited to only one release of the drug in the body, but can continuously release the drug while staying in vivo two or more times as necessary.

In particular, considering that such action can be implemented at a thickness of about 500 μm or less, ultra-thin thickness can be achieved through coplanar arrangement of the aperture and the intermediate outlet, horizontal movement structure of a drug, use of one membrane, suppression of interference between membranes using a fixed area, and the like.

In addition, releasing amount and timing of a drug can be determined by adjusting the strength of the magnetic field or the exposure time to the magnetic field, which can therefore prevent excessive drug injection from causing toxicity to surrounding tissues, and conversely, if necessary, the dose can be increased to deliver a high dose of a drug to maximize therapeutic effect.

### BRIEF DESCRIPTION OF DRAWINGS

FIG. 1 is a view for illustrating the use of an ultra-thin drug pump that can be installed into a lens according to an embodiment of the present disclosure.

FIG. 2 is a cross-sectional view for illustrating the structure of an ultra-thin drug pump according to an embodiment of the present disclosure.

FIG. 3 is a plan view for illustrating the structure of an ultra-thin drug pump of FIG. 2.

FIG. 4 is an exploded view for illustrating the structure of an ultra-thin drug pump of FIG. 2.

FIGS. 5A and 5B illustrate operating states of an ultra-thin drug pump of FIG. 2.

FIG. 6 shows photographs listing a process of releasing a drug from an ultra-thin drug pump according to an embodiment of the present disclosure.

FIGS. 7A-7C illustrate a process of bonding a membrane and a third layer in an ultra-thin drug pump according to an embodiment of the present disclosure.

FIGS. 8A-8D show graphs for estimating a variation in a drug chamber according to dimensions of an aperture and dimensions of a micro-driving part in an ultra-thin drug pump according to an embodiment of the present disclosure.

FIGS. 9A-9C shows plan photographs of an ultra-thin drug pump according to an embodiment of the present disclosure.

FIGS. 10A-10C show side photographs of an ultra-thin drug pump according to an embodiment of the present disclosure.

FIG. 11 is a graph showing a release rate according to a magnetic field strength in an ultra-thin drug pump according to an embodiment of the present disclosure.

FIG. 12 is a cross-sectional view for illustrating the structure of an ultra-thin drug pump according to an embodiment of the present disclosure.

FIG. 13 is a plan view for illustrating the structure of an ultra-thin drug pump of FIG. 12.

### DETAILED DESCRIPTION

Hereinafter, preferred embodiments of the present disclosure will be described in detail with reference to the accompanying drawings, but the present disclosure is not limited or restricted by the embodiments. For reference, in the detailed description, like numbers refer to substantially the same or similar elements, and contents described in other drawings under the above rules may be cited and described, and descriptions repeated or apparent to those skilled in the art may be omitted.

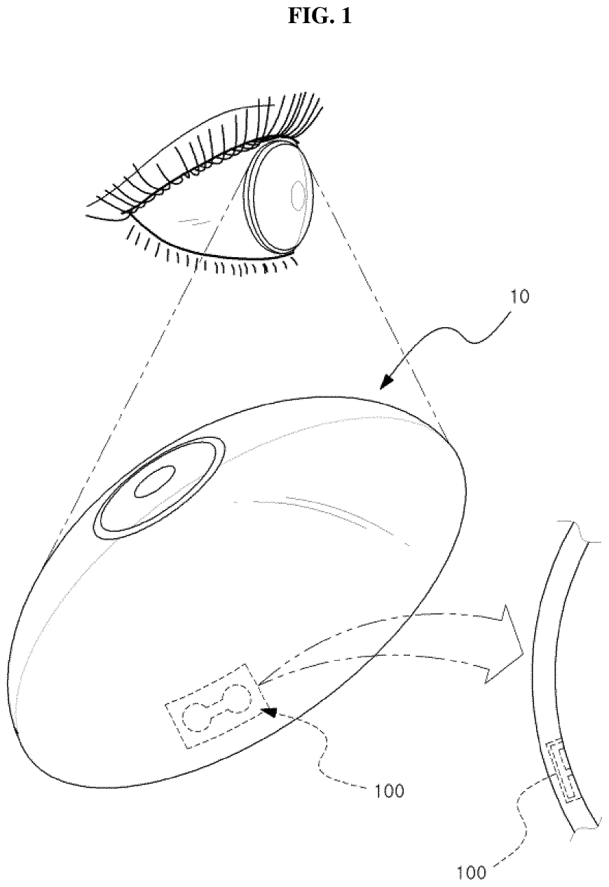

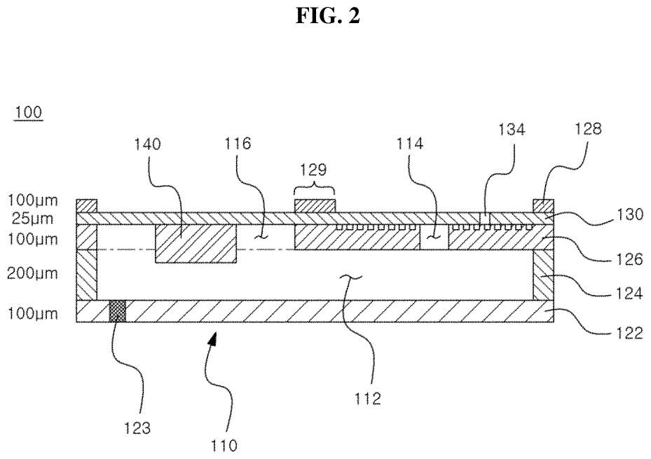

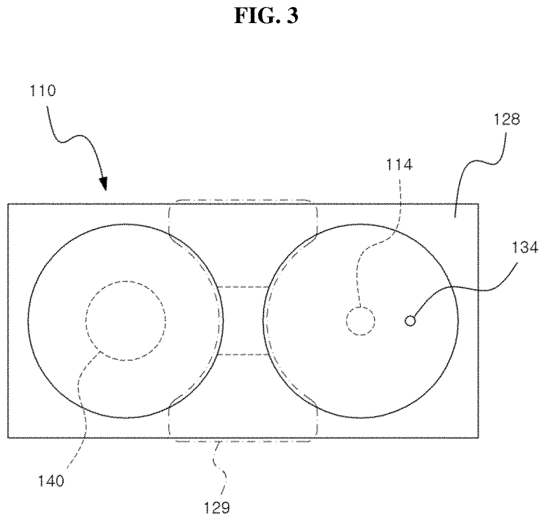

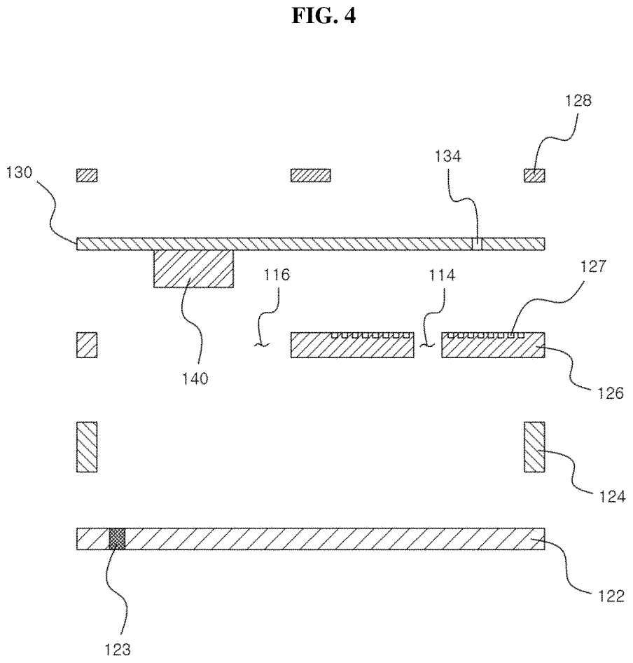

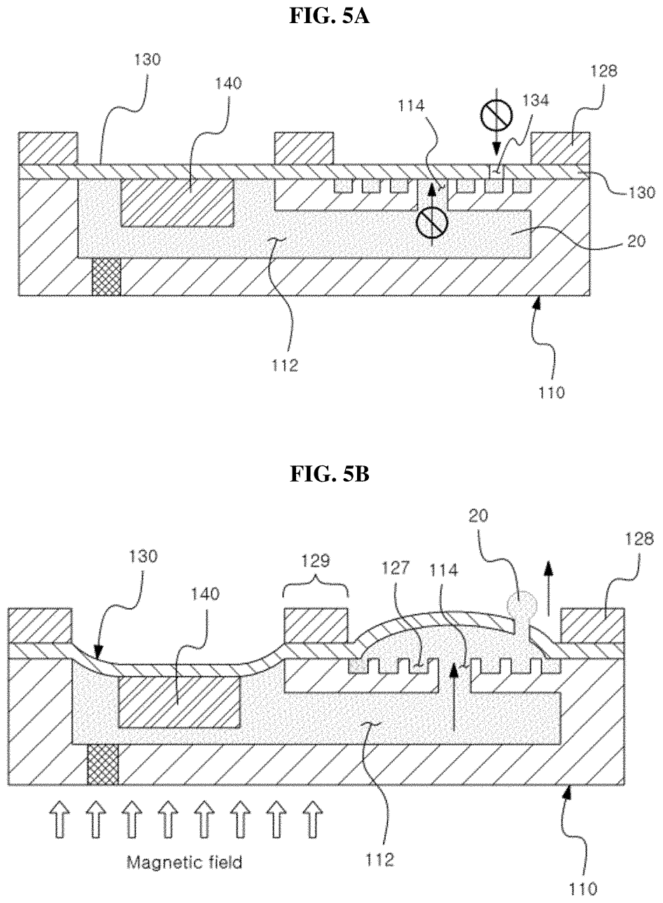

FIG. 1 is a view for illustrating the use of an ultra-thin drug pump that can be installed into a lens according to an embodiment of the present disclosure; FIG. 2 is a cross-sectional view for illustrating the structure of an ultra-thin drug pump according to an embodiment of the present disclosure; FIG. 3 is a plan view for illustrating the structure of an ultra-thin drug pump of FIG. 2; FIG. 4 is an exploded view for illustrating the structure of an ultra-thin drug pump of FIG. 2; and FIGS. 5A and 5B illustrate operating states of an ultra-thin drug pump of FIG. 2.

Referring to FIG. 1, the ultra-thin drug pump (100) according to one embodiment of the present disclosure may be installed into a thin structure such as a contact lens (10). Although the contact lens is illustrated in this embodiment, the contact lens may be fixedly mounted on another structure, a tissue of a body, or a blood vessel wall using a thin thickness.

When the ultra-thin drug pump (100) is mounted on the contact lens (10), the user may wear the contact lens provided with the ultra-thin drug pump, and when fixed to a part of a body, one procedure or surgery may be required for the user.

The ultra-thin drug pump (100) may be formed of a material such as polyester or polyurethane that can be biodegradable in vivo. In some cases, however, if it is necessary to stay for a long time, such as 1 to 2 years or more, it may be formed of a material that is not biodegradable, in which case a separate surgery may be required to remove the drug pump (100).

In particular, it is necessary to manufacture a thin and hard drug pump for inserting a contact lens, etc. For this purpose, several biocompatible materials, such as polydimethylsiloxane (PDMS), polyurethane acrylate (PUA), Ostemers, etc., may be exemplified. PUA is cured with UV and has a high Young's modulus (e.g., about 19.8 MPa), but the toughness is poor and may be easy to be broken. Ostermers™ 322 crystal clear is harder than PDMS or PUA and has the advantage of providing a desired shape through UV and heat when cured. Young's modulus of Ostermers™ is about 60 MPa when first cured, and about 1 GPa when fully cured.

For reference, the ultra-thin drug pump according to one embodiment of the present disclosure may be applied to a contact lens prepared by a sandwiching process disclosed in Korean Patent No. 10-0647133.

Referring to FIG. 2, the ultra-thin drug pump (100) includes a micro-body (110) including a drug chamber (112), an intermediate outlet (114) formed on one upper side and an aperture (116) formed on the other upper side; a membrane (130) covering the intermediate outlet (114) and the aperture (116); and a magnetic driving part (140) formed on the membrane (130).

The aperture (116) and the intermediate outlet (114) may be covered by one membrane (130), and a drug outlet (134) may be formed at a position spaced apart from the intermediate outlet (114) in the membrane (130). The membrane (130), although it is one element, may function in various ways in the drug pump (100) of this embodiment. For example, the membrane (130) can exert the following functions of: covering the aperture (116) of the drug chamber (112) together with the drug chamber (112); moving by the magnetic driving part (140) to pressurize a drug in the drug chamber (112); blocking the intermediate outlet (114) formed on an upper side of the micro-body (110); and releasing a drug through the drug outlet (134) while being partially deformed when a pressure equal to or greater than a predetermined pressure is formed in the drug chamber (112).

Preferably, the micro-body (110) of the drug pump (100) may be made using Ostemers™ 322 crystal clear to form a thin and rigid body, and the membrane (130) may be formed using PDMS because of its relatively flexible nature. In addition, the micro-body or the membrane may be formed using other properties of PDMS, hydrogel structure, silicone polymer, and the like. The micro-body or the membrane may be formed through a soft lithography process. They may be formed using biodegradable or non-biodegradable materials depending on the period of time it is harmless in vivo and is required to be maintained in vivo.

The micro-body (110) and the membrane (130) may form a temporarily sealed drug chamber (112). In the drug chamber (112), the drug (20) may enter through an inlet (123), and the drug (20) may be stored while the drug pump (100) operates. As described above, the stored drug (20) may be released to the outside through the intermediate outlet (114) formed on one side of the micro-body (110).

In this embodiment, the aperture (116) on which the membrane (130) is formed and the intermediate outlet (114) through which the drug (20) passes are formed in the same direction.

The membrane (130) may be made using PDMS, etc., and formed with a relatively thin thickness (e.g., about 25 μm) as compared to the layer of the micro-body (110) (e.g., about 100 μm). The membrane (130) may be deformed prior to the deformation of the micro-body (110) to maintain the shape of the micro-body (110) and the shape of the drug chamber (112).

In this embodiment, the micro-body (110) may be formed using multiple layers of Ostemers. Specifically, referring to FIG. 4, the micro-body (110) may include , from the bottom thereof, a first layer (122) forming a bottom; a second layer (124) forming an outermost sidewall for the drug chamber (112) on the first layer (122); a third layer (126) provided on the second layer (124) and including an aperture (116) and an intermediate outlet (114); and a fourth layer (128) stacked over a membrane (130) with the membrane (130) interposed therebetween.

For reference, the first layer (122), the third layer (126) and the fourth layer (128) may be formed in a thickness of about 100 and the second layer (124) may be formed in a thickness of about 200 μm up and down, and the membrane (130) may be formed in a thickness of about 25 μm. When forming using a mold, the second layer and the third layer may be integrally formed together.

In this embodiment, the fourth layer (128) may cover a fixed area (129), a periphery of the aperture (116), and a periphery of the intermediate outlet (114), and the third layer (126) and the fourth layer (128) may prevent deformation of some of the membrane (130) from affecting other functions. That is, the membrane (130) may be fixed to the micro-body (110) by the third layer (126) and the fourth layer (128), and, in the fixed area (129), the membrane (130) may be secured by the third and fourth layers so that deformation of a portion of the membrane around the aperture (116) may not affect the membrane (130) around the intermediate outlet (114), and deformation of the portion of the membrane around the intermediate outlet (114) may not affect the membrane (130) around the aperture (116).

Referring to FIG. 5A, the membrane (130) restricts the movement of the drug between the intermediate outlet (114) and the drug outlet (134) while the drug pump (100) is not operated, thereby preventing leakage through diffusion, and the membrane (130) may close the aperture (116) of the micro-body (110) to prevent foreign substances from entering the drug chamber (112) or the drug of the chamber from leaking to the outside.

Referring to FIG. 5B, when magnetic field is applied, a magnetic driving part (140) may move downward, and a portion of the membrane (130) along with the magnetic driving part (140) may be operated downward, so that the pressure in the drug chamber (112) increases, whereas a portion of the membrane (130) positioned on the opposite side may swell upward, so that the intermediate outlet (114) and the drug outlet (134) are opened, and the drug may be released to the outside.

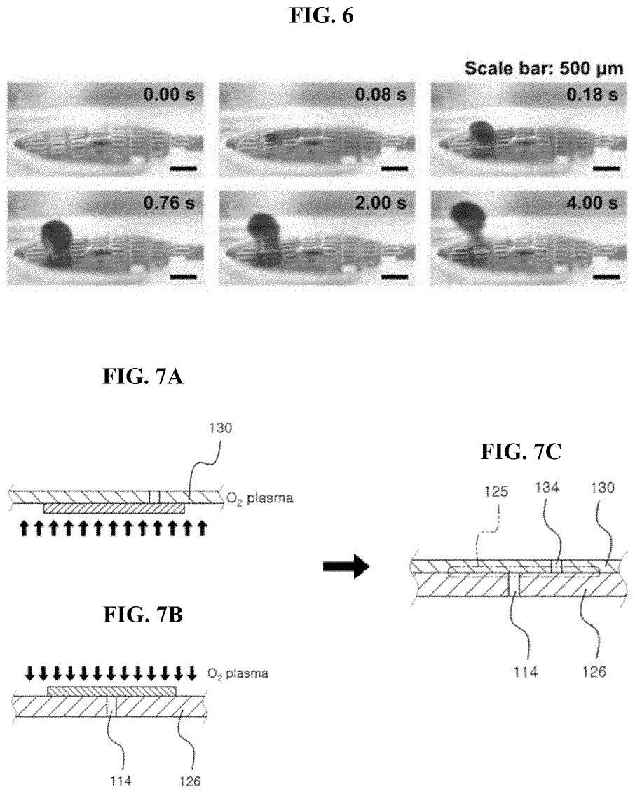

FIG. 6 shows photographs listing a process of releasing a drug from an ultra-thin drug pump according to an embodiment of the present disclosure.

Referring to FIG. 6, when no pressure is applied, the membrane is not inflated (0.00 s), and when pressure is applied, a part of the membrane is inflated and some of the drug may begin to be released through the drug outlet formed at the left side (0.18 s). When a predetermined amount of drug is released as the pressure is gradually increased by the magnetic driving part (0.20 s), only a predetermined amount of drug can be released to the outside while the drug outlet is closed again (4.00 s). For reference, the photos are taken at an interval of about 0.02 seconds, and show what is happening under a magnetic field.

Referring back to FIGS. 2 and 4, it can be seen that a non-bonded region (125) is formed between the third layer (126) and the membrane (130) around the intermediate outlet (114). The non-bonded region (125) is to prevent deadlock between the third layer (126) and the membrane (130), and, in this embodiment, a plurality of grooves (127) may be formed to correspond to the non-bonded region (125).

The plurality of grooves (127) may be formed in a shape of a lattice having a depth of about 25 μm or less from the micro-body (110) around the intermediate outlet (114), and, through this patterning, it is possible to solve the problem of the membrane (130) adhering to an outer surface of the micro-body (110).

The membrane (130) and the micro-body (110) form a non-bonded region (125) around the intermediate outlet (114) so that the membrane (130) may be partially deformed, and the drug outlet (134) may be formed at one side of the region where deformable. The drug outlet (134) may be spaced apart from the intermediate outlet (114), and the release amount of the drug released once may be controlled by the size of the drug outlet (134), the distance separated, and the restoring force of the membrane (130).

The magnetic driving part (140) may be integrally coupled to the membrane (130). In this embodiment, the magnetic driving part (140) may be formed by mixing PDMS and magnetic nanoparticles, and may move in one direction by a magnetic field. In addition, it is formed at a center of the aperture (116), may be provided to be formed inside the membrane (130) so as not to protrude to the outside.

The drug pump (100) according to this embodiment may be manufactured based on a soft lithography process, and the magnetic driving part (140) may be formed through a magnetic composite polymer (MCP) membrane in which magnetic nanoparticles are combined with a flexible PDMS material.

In order to manufacture a drug pump made of Ostemer/PDMS materials, a first part including the fourth layer (128), the magnetic driving part (140), and the membrane (130) is manufactured, and a second part including the first layer (122) to the third layer (126) is manufactured, and then the first part and the second part may be assembled to complete an assembly of the micro-body (110) and the membrane (130). The aperture (116), the intermediate outlet (114), the drug outlet (134), and the inlet (123) may be formed through laser cutting, and the drug may be injected into the pump using a syringe and a microneedle through the inlet (123), and the inlet (123) may be sealed with PDMS to complete the drug pump (100).

The magnetic driving part (140) may be formed by coating a PDMS (polydimethylsiloxane) layer for the membrane (130) by spin coating on the substrate, and then spin coating a magnetic-PDMS layer for the magnetic driving part (140) over another substrate, and then punching it according to a pattern. In this embodiment, a silane-treated silica may be used as the substrate, and the magnetic-PDMS layer may be manufactured by mixing 1:1 of Ferrotec's EMG 1200 magnetic nanoparticles and Sylgard™ 184 PDMS. In addition, the magnetic driving part (140) punched on the PDMS layer may be bonded, and integrally contacted with the fourth layer (128).

FIGS. 7A-7C illustrate a process of bonding a membrane and a third layer in an ultra-thin drug pump according to an embodiment of the present disclosure.

Referring to FIG. 7, a mask using PDMS may be stacked on the third layer (126) in which the intermediate outlet (114) is formed to correspond to the non-bonded region (125), and may be treated with oxygen (O₂) plasma. Further, a mask using PDMS may also be stacked on the membrane (130) in which the drug outlet (134) is formed to correspond to the non-bonded region (125), and may also be treated with oxygen (O₂) plasma.

Therefore, in the process of bonding the third layer (126) and the membrane (130), the non-bonded region (125) may be formed around the intermediate outlet (114), and the movement of the relatively thin membrane (130) allows the membrane (130) to operate smoothly as a sort of valve.

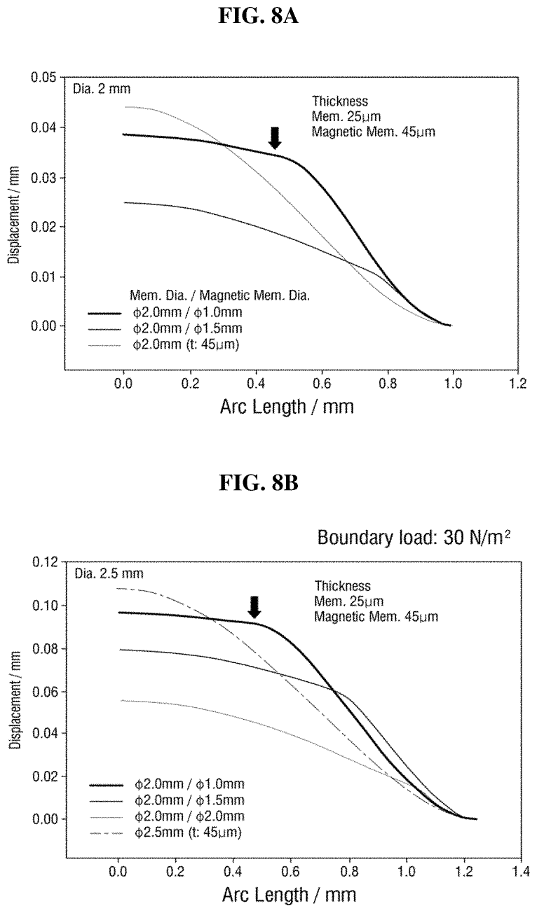

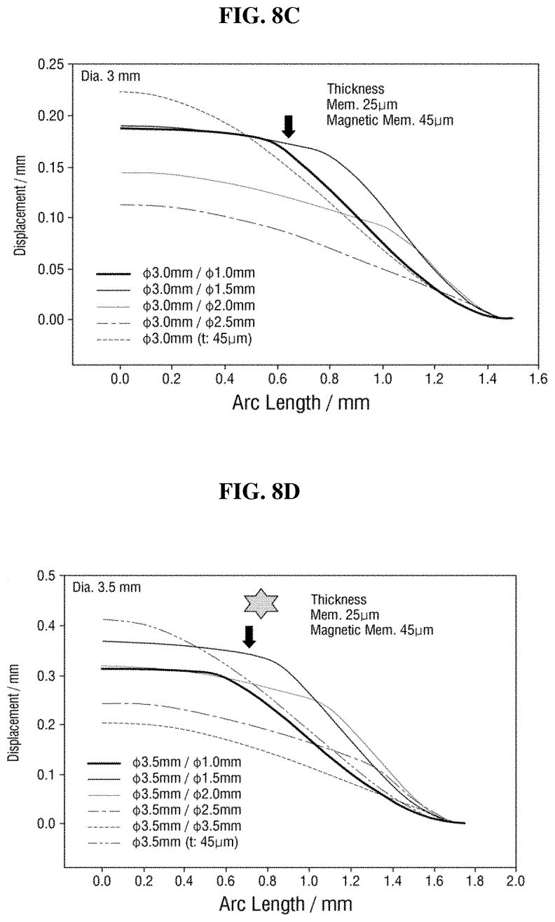

FIGS. 8A-8D show graphs for estimating a variation in a drug chamber according to the dimensions of an aperture and the dimensions of a micro-driving part in an ultra-thin drug pump according to an embodiment of the present disclosure.

Since the drug pump according to this embodiment is required to be thin enough to be inserted into a contact lens, this size limitation necessitates optimizing the dimensions in advance to achieve a quantitative goal. To this end, the magnetic driving part and the membrane may be designed using COMSOL simulation programs to optimize their size.

Referring to FIG. 8, the simulation was performed by varying the diameters of the membrane and the magnetic driving part. For example, the diameters of the membrane varied to 2.0 mm, 2.5 mm, 3.0 mm, and 3.5 mm, and, in each membrane, the magnetic driving part was placed in the center and the diameters thereof varied to 1.0 mm, 1.5 mm, 2.0 mm, 2.5 mm, and 3.0 mm. The thickness of the membrane was 25 μm, and the thickness of the magnetic driving part was 45 μm.

Simulation results show that the driving displacement tends to increase as the diameter of the membrane increases, and thus, when the diameter of the membrane showing maximum displacement was 3.5 mm and the diameter of the magnetic driving part was 1.5 mm, the change in volume was greatest. In addition, the ratio of the diameter of the magnetic driving part to the diameter of the membrane showed the best volume change in a range of 0.42 to 0.60.

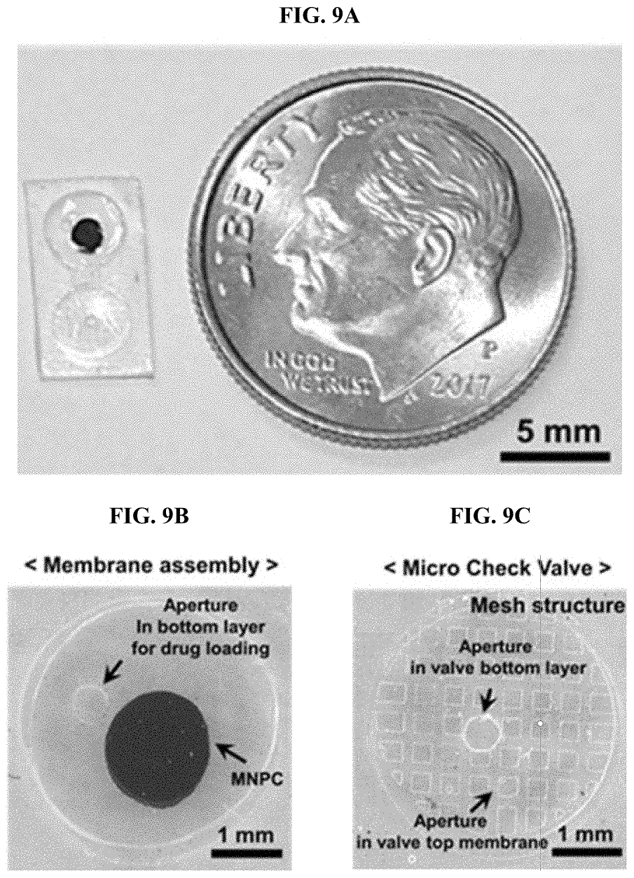

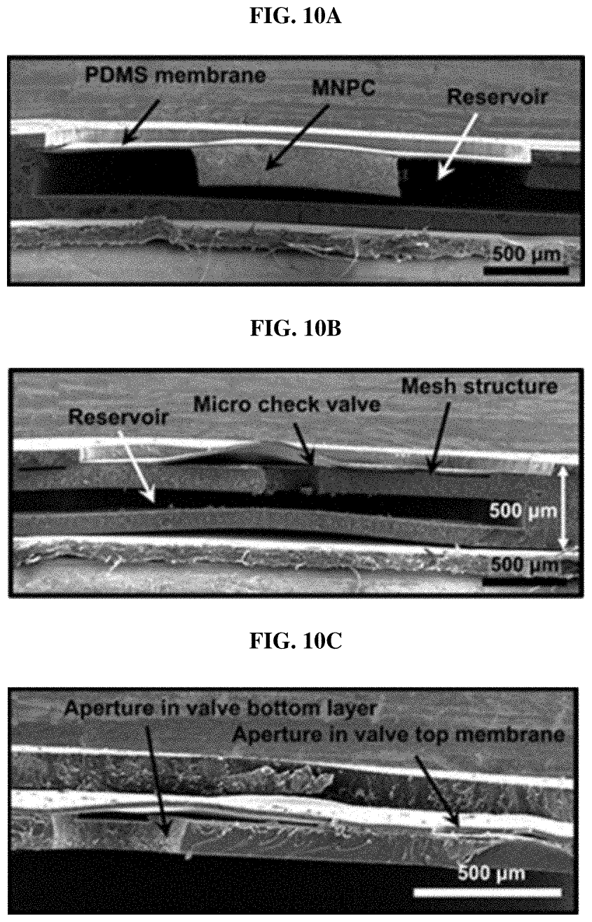

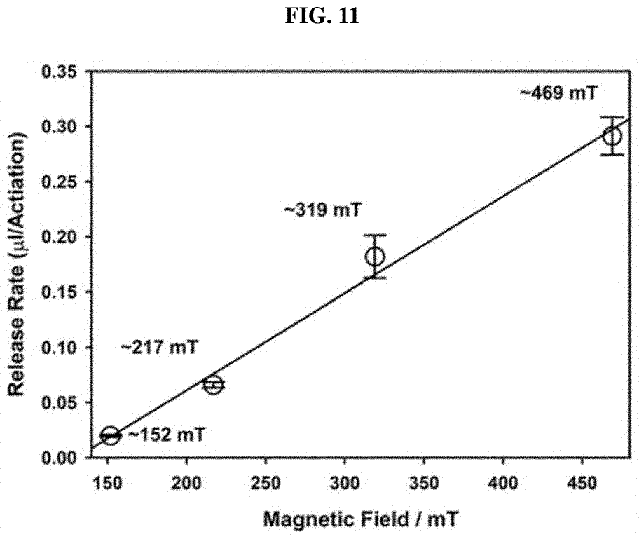

FIGS. 9A-9C show plan photographs of an ultra-thin drug pump according to an embodiment of the present disclosure, and FIGS. 10A-10C show side photographs of an ultra-thin drug pump according to an embodiment of the present disclosure.

Referring to FIGS. 9 and 10, the micro-body having the drug chamber formed in a lower portion of the membrane is provided, and the magnetic driving part is integrally formed on the bottom of the membrane. In the planar view, the intermediate outlet and the drug outlet may be formed at positions spaced apart from each other, and mesh-structured grooves may be formed on the outer surface of the micro-body to prevent deadlocks between the membrane and the micro-body.

For reference, the size of the membrane exposed by the aperture is about 3.5 mm, the diameter of the drug outlet is about 350 μm, and the diameter of the intermediate outlet is about 500 μm. Since the thickness of the drug pump was about 500 μm or less, the drug pump was sized enough to be inserted into a contact lens. In this case, the total drug content of the drug pump is about 4.5 μl.

FIG. 11 is a graph showing a release rate according to a magnetic field strength in an ultra-thin drug pump according to an embodiment of the present disclosure.

Referring to FIG. 11, a release amount of the drug pump according to a magnetic field strength may be measured through an image. The graph indicates that when the magnetic field strength was about 152 mT, the amount of drug release was about 0.02 μl; when the magnetic field strength was about 217 mT, the amount of drug release was about 0.07 μl; when the magnetic field strength was about 319 mT, the amount of drug release was about 0.18 μl; and when the magnetic field strength was about 469 mT, the amount of drug release was about 0.29 μl.

In addition, the change in the amount of drug released according to the number of operation of the drug pump was measured. The measurement results showed that the amount of drug release of the drug pump remains almost constant in the magnetic field of about 152 mT to about 217 mT.

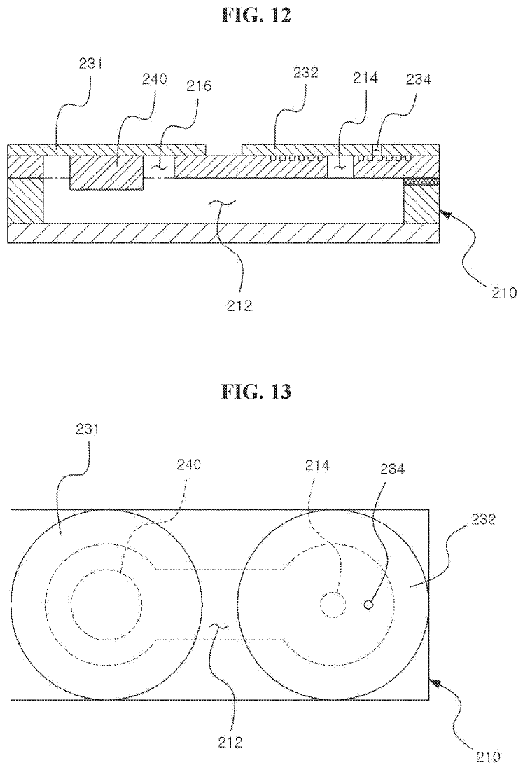

FIG. 12 is a cross-sectional view for illustrating the structure of an ultra-thin drug pump according to an embodiment of the present disclosure, and FIG. 13 is a plan view for illustrating the structure of an ultra-thin drug pump of FIG. 12.

Referring to FIGS. 12 and 13, the ultra-thin drug pump (200) may include a first membrane (231) covering a drug chamber (212) and an aperture (216); a second membrane (232) covering an intermediate outlet (214) and including a drug outlet (234) formed at a position spaced apart from the intermediate outlet (214); and a magnetic driving part (240) formed on the first membrane (231).

Unlike the previous embodiment, in this embodiment, the membrane may be separated to a first membrane (231) and a second membrane (232), which may cover the aperture (216) and the intermediate outlet (214), respectively.

The first membrane (231) can serve to cover the aperture (216) of the drug chamber (212) together with the drug chamber (212), and to move by a magnetic driving part (240) to pressurize a drug in the drug chamber (212), and the second membrane (232) can function to block the intermediate outlet (214) formed on one upper side of the micro-body (210), and to release the drug through the drug outlet (234) while being partially deformed when a pressure equal to or greater than a predetermined pressure is formed in the drug chamber (212).

Since the first membrane (231) and the second membrane (232) are separated from each other, even if the fixed area is not formed separately, the first membrane (231) and the second membrane (232) may not be mutually influenced even when they move in opposite directions.

As described above, although the present disclosure is described with reference to a preferred embodiment thereof, those skilled in the art will appreciate that various modifications and changes can be made in the present disclosure without departing from the spirit and scope of the invention as set forth in the claims below.

What is claimed is:

1. An ultra-thin drug pump comprising:
a micro-body comprising a drug chamber, an intermediate outlet formed on one upper side, and an aperture formed on the other upper side;
a first membrane covering the aperture;
a second membrane covering the intermediate outlet and comprising a drug outlet formed at a position spaced apart from the intermediate outlet; and
a magnetic driving part formed on the first membrane,
wherein as the magnetic driving part moves in one direction by a magnetic field, the first membrane pressurizes a drug in the drug chamber, and then some of the drug is moved between the second membrane and an upper surface of the micro-body through the intermediate outlet, and the second membrane is partially deformed to release the drug through the drug outlet.

2. The ultra-thin drug pump of claim 1, wherein the first membrane and the second membrane is formed with the same membrane.

3. The ultra-thin drug pump of claim 2, further comprising a fixed area in which a portion of the membrane between the aperture and the intermediate outlet is secured to an upper surface of the micro-body to block a membrane deformation at the aperture and a membrane deformation at the intermediate outlet.

4. The ultra-thin drug pump of claim 3, wherein the micro-body comprises a first layer forming a bottom; a second layer forming an outermost sidewall for the drug chamber on the first layer; a third layer provided on the second layer and including the aperture and the intermediate outlet; the membrane formed on the third layer; and a fourth layer provided on the membrane and covering a periphery of the aperture and a periphery of the intermediate outlet including the fixed area, and
wherein the membrane is secured to the micro-body by the third and fourth layers, and, in the fixed area, the membrane is secured by the third and fourth layers, such that deformation of a portion of the membrane around the aperture is not transferred to the membrane around the intermediate outlet.

5. The ultra-thin drug pump of claim 4, wherein, in the third layer, a non-bonded region is formed at a periphery of the intermediate outlet.

6. The ultra-thin drug pump of claim 5, wherein a plurality of grooves is formed on an upper surface of the third layer in contact with the membrane corresponding to the non-bonded region.

7. The ultra-thin drug pump of claim 5, wherein a bottom surface of the membrane and an upper surface of the third layer corresponding to the non-bonded region are masked and exposed to oxygen plasma.

8. The ultra-thin drug pump of claim 1, wherein the magnetic driving part is located at a center of the diameter of the aperture, and
the diameter of the magnetic driving part relative to the diameter of the aperture is in the range of 0.42 to 0.60.

9. The ultra-thin drug pump of claim 1, wherein the micro-body is provided using Ostemers™, and the membrane is provided using PDMS.

10. The ultra-thin drug pump of claim 1, wherein the magnetic driving part is provided by stirring PDMS and magnetic nanoparticles.

11. The ultra-thin drug pump of claim 1, wherein the magnetic driving part is formed to face the drug chamber at a bottom surface of the first membrane.
